(19) United States
(12) Patent Application Publication
(10) Pub. No.: US 2002/0177891 A1
(43) Pub. Date: Nov. 28, 2002

(54) Endoluminal device and method for fabricating same

(76) Inventor: Juan Carlos Parodi, Buenos Aires (AR)

Correspondence Address: Patrick W. Rasche, Armstrong Teasdale LLP, One Metropolitan Sq., Suite 2600, St. Louis, MO, 63102, US

(21) Appl. No.: 10/133,969

(22) Filed: Apr. 26, 2002

Related U.S. Application Data
(60) Provisional application No. 60/286,551, filed on Apr. 26, 2001.

(51) Int. Cl. A61F 2/06

(52) U.S. Cl. 623/1.15

(57) ABSTRACT

An endovascular device is configured to elastically expand to a first outer diameter, plastically deform to a second outer diameter, and retain a third outer diameter that is greater than about 90% of the second outer diameter after a device that has been utilized to deform the endovascular device to the second outer diameter has been removed from the endovascular device. A method of fabricating the endovascular device includes aging the endovascular device at about 485° C. for about 120 minutes.

### CROSS REFERENCE TO RELATED APPLICATIONS

This application claims the benefit of U.S. Provisional Application No. 60/286,551 filed Apr. 26, 2001, which is hereby incorporated by reference in its entirety.

### BACKGROUND OF THE INVENTION

This invention relates generally to endoluminal devices and, more specifically, to the manufacture and use of endoluminal devices that have combined elastic and plastically deformable properties.

Endoluminal devices comprise any type of medical device, such as but not limited to stents, grafts, prostheses, vena cava filters, and the like, that are inserted into body lumen. A stent is an elongated device used to support a vessel wall. Stents are currently used in a large variety of pathological conditions usually to expand a narrowing of a hollow vessel such as an artery or esophagus. As an example, a stent provides an unobstructed conduit for blood in the area of a stenosis. Stents, in comparison to balloon angioplasty alone, keep a lumen of the hollow vessel open counteracting the elastic recoil of the vessel. Known stents are typically either plastically deformable and balloon-expandable, such as stents made of stainless steel, or elastic and self-expandable.

In addition, stents may also include a prosthetic graft layer of fabric or covering lining the inside or outside thereof. Such a covered stent is commonly referred to in the art as an intraluminal prosthesis, an endoluminal or endovascular graft (EVG), or a stent-graft. Intraluminal prostheses are typically used to re-line an artery, such as for treatment of dilatations of the arteries (aneurysms).

A prosthesis may be used, for example, to treat a vascular aneurysm by removing the pressure on a weakened part of an artery which reduces the risk of rupture. Typically, a prosthesis is implanted in a blood vessel at a site of a stenosis or aneurysm endoluminally, i.e. by so-called “minimally invasive techniques” in which the prosthesis, restrained in a radially compressed configuration by a sheath or catheter, is delivered by a deployment system or “introducer” to the site where it is required. The introducer may enter the body through the patient's skin, or by a “cut down” technique in which the entry blood vessel is exposed by minor surgical means. When the introducer has been threaded into the body lumen to the prosthesis deployment location, the introducer is manipulated to cause the prosthesis to be ejected from the surrounding sheath or catheter in which it is restrained. Alternatively the surrounding sheath or catheter is retracted from the prosthesis. Upon release of the prosthesis, the prosthesis expands to a predetermined diameter at the deployment location, and the introducer is withdrawn. The expansion of the prosthesis may be effected by spring elasticity, plastic deformation, or by die self-expansion of a thermally or stress-induced return of a memory material to a pre-conditioned expanded configuration.

Typically, endoluminal devices such as stents expand by one mechanism or another, not by a combination of mechanisms. That is, plastically deformable devices are not typically elastic, and elastic devices are not typically plastically deformable

### BRIEF DESCRIPTION OF THE INVENTION

In accordance with one aspect of the invention, a method is provided for making an endoluminal device including at least one superelastic section and at least one plastically deformable section. The superelastic section and plastically deformable section comprise a continuous metallic structure in which the superelastic section has been thermally treated differently than the plastically deformable section. The method comprising selecting a metallic member to be used for constructing the endoluminal device, heat treating at least a first portion of the metallic member in a first annealing step under a first set of conditions to set a shape memory for at least the first portion, heat treating one or more second portions of the metallic member in a second annealing step under a second set of conditions to make the second portions plastically deformable, and forming the metallic member into the endoluminal device such that the first portion comprises the superelastic section and the second portion comprises the plastically deformable section.

In another aspect of the invention, an endovascular device is provided that is configured to elastically expand to a first outer diameter, plastically deform to a second outer diameter, and retain a third outer diameter that is greater than about 90% of the second outer diameter after a device that has been utilized to deform the endovascular device to the second outer diameter has been removed from the endovascular device.

In another aspect of the invention, a method is provided to heat treat a hybrid endovascular device that is both elastic and plastically deformable. The method comprises aging the endovascular device at about 485° C. for about 120 minutes.

In another aspect of the invention, a method is provided for deploying a hybrid endovascular device fabricated from a single composition and that has undergone a single heat treatment. The method comprises positioning a hybrid endovascular device on an introducer, introducing the endovascular device to the proper position, allowing the endovascular device to elastically expand to a first outer diameter, and plastically deforming the endovascular device to a second outer diameter.

### BRIEF DESCRIPTION OF THE DRAWINGS

FIG. 1 illustrates an exemplary tube of superelastic material undergoing a first annealing step.

FIG. 2 illustrates the tube shown in FIG. 1 after an exemplary second annealing step.

FIG. 3 illustrates an endograft formed after a precision cutting step performed on the tube of FIG. 2 and after attachment of a graft liner.

### DETAILED DESCRIPTION OF THE INVENTION

Exemplary embodiments of endoluminal devices and methods of fabricating the devices are described below. In one embodiment, an elastic and plastically deformable endoluminal device is fabricated from a nitinol compound. The nitinol compound is used to form an endoluminal device, such as a stent, that is heat treated to create a material having both elastic and plastically deformable properties. Although exemplary embodiments are described herein, the endoluminal device and methods are not limited to those specific embodiments.

The endoluminal device and method are illustrated with reference to the figures wherein similar numbers indicate the same elements in all figures. Such figures are intended to be illustrative rather than limiting and are included herewith to facilitate the explanation of an exemplary embodiment of the device and method of the invention.

The term “endoluminal device” is used herein to refer to any type of implantable device, such as but not limited to stents, grafts, prosthesis, and vena cava filters, which may benefit from the following teaching. Although the exemplary embodiments described and illustrated herein refer specifically to stents, the following teachings should not be interpreted to be limited to only stents. Thus, descriptions of stents should be considered applicable to other endoluminal devices, where applicable.

In an exemplary embodiment, an endoluminal device is both plastically deformable and superelastic. As used herein, a “superelastic” material is one that may be deformed into a certain configuration without the material permanently taking on the deformed shape. For example, a thermal shape memory material may be deformed into a number of configurations, but will return to its memory shape upon temperature activation. A “plastically deformable” material, on the other hand, is a material that once deformed into a certain shape, keeps that shape indefinitely, until deformed again by some other force.

In general, endoluminal devices are typically inserted into a body lumen from a remote location through an insertion point in the body through which an “introducer,” containing the device in a radially compressed configuration, is threaded and navigated through the body lumen to the deployment location, where the device is deployed in a radially expanded configuration. As referred to herein, “distal” refers to the direction further away from the insertion point and “proximal” refers to the direction closer to the insertion point.

The combination of plastically deformable and superelastic properties provide unique characteristics that allow good trackability (ease of maneuvering through tortuous lumen), good flexibility, a low profile, good conformability and high radial force. There are several methods to obtain different characteristics in a single endoluminal device:

1) Different materials are joined in a single unit by welding, crimping or any other method of attaching to join the parts and keep the components together. Exemplary methods are explained in detail in U.S. patent application Ser. No. 09/702,226, filed on Oct. 31, 2000, by Steven E. Walak, incorporated herein by reference.

2) The same materials with quantitative different proportions of individual components (e.g. nitinol with more nickel and less titanium) are used, such as is also described in the '226 application.

3) Differential thermal treatment of different parts of a single compositional unit of a device, such as a device comprising nitinol, is performed. There are at least two ways to treat the nitinol material to achieve the desirable differential characteristics: treating the final designed device, or treating the nitinol before constructing or building the device. Materials other than nitinol having similar characteristics may be similarly treated. Methods of first fabricating the device and then thermally treating it are described in detail in U.S. patent application Ser. No. 09/362,261, filed on Jul. 28, 1999, by Steven E. Walak and Paul DiCarlo, incorporated herein by reference. The method of using thermal differential treatment to treat the material before constructing or building the stent is described and claimed in this application.

4) A stent is fabricated from a single compositional material utilizing a single heat treatment process applied to the entire stent.

An exemplary embodiment of an endoluminal device comprises a one-piece stent made of a hollow tube of nitinol that is then treated by laser energy to create the design of the stent. While laser treatment is a common method for performing a cutting step to create the design, as is known in the art, the method is not limited to any particular cutting mechanism, and may instead comprise another chemical or mechanical precision cutting step, such as but not limited to chemical etching. To create both plastically deformable and superelastic regions in the same stent, different regions of the hollow tube are thermally treated differently in accordance with available technology, as described below, prior to the cutting step.

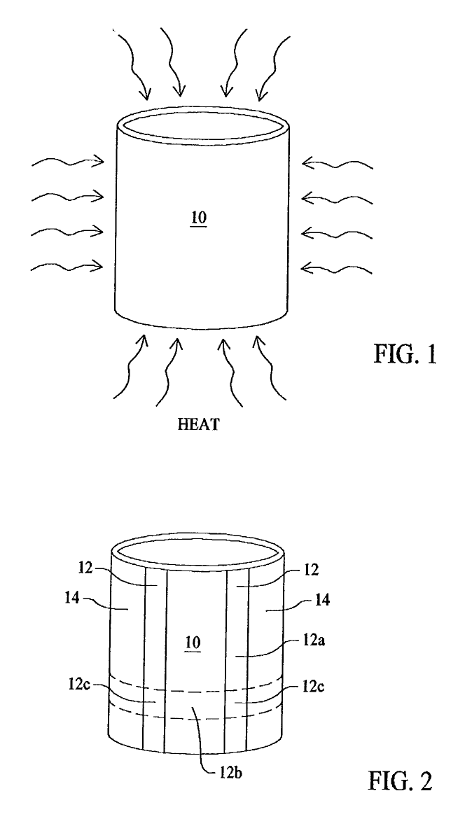

It is known that with sufficient heat treatment, superelastic properties of shape-memory material such as nitinol may be destroyed. Referring now to FIGS. 1-3, an exemplary method is initiated by selecting a tube 10 comprising a superelastic material, such as nitinol. Tube 10 is typically subject to a first annealing step under standard conditions known in the art, as shown in FIG. 1. In one embodiment, the material is annealed for about zero minutes to about 60 minutes at a temperature ranging from about 400° C. to about 600° C. More particularly, the material is annealed for about 5 minutes to about 40 minutes at a temperature ranging from about 400° C. to about 575° C. More particularly still, the device is annealed for about 10 minutes to about 15 minutes at a temperature ranging from about 450° C. to about 550° C. In an alternative embodiment, tube 10 is not subjected to a first annealing step. In further alternative embodiments, shorter or longer periods and/or higher or lower temperatures may be used depending on the material being annealed and the properties desired. The first annealing process is used to set the memory shape to which the material will return.

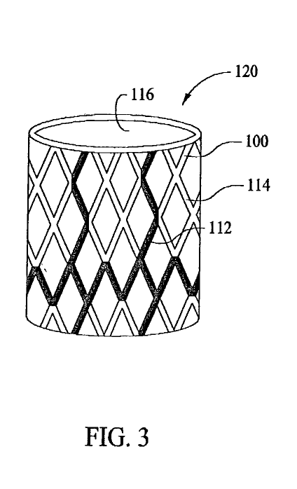

After the first annealing step, selected portions 12 of tube 10 are subjected to a second, localized, annealing step in which the portions are heated further. Although selected portions 12 are shown in FIG. 2 as vertical stripes 12a and horizontal stripes 12b, the selected portions may comprise horizontal stripes only, vertical stripes only, or isolated areas such as regions 12c at the intersections of vertical stripes 12a. Selected portions 12 may have any geometric shape desired, as may isolated areas, and the selected portions or isolated areas may be randomly or orderly spaced at any interval desired to provide a desired ratio of plastically deformable to superelastic area.

The localized heat treatment in the second annealing step may be accomplished by any suitable method. One such method includes the use of electrical resistance heating. Electrical leads are attached across the desired portions of tube 10 and a current is allowed to pass therethrough. Because of the resistance of the shape-memory metal, the desired portion of metal heats up, further annealing the material. Another suitable method comprises applying a heated inert gas jet to desired portions of tube 10 to selectively heat those portions. Another method includes induction coil heating wherein an induction coil is placed over desired portions of tube 10 to effect induction heating of the desired portions of tube 10. Laser heating, in which a laser is used to selectively heat desired regions of the device, may also be performed. The desired regions may also be heated by brazing. Furthermore, the device may be placed in a fluidized bath of a heat-treating fluid such as a salt bath or a fluidized sand bath, with appropriate sections of the tube insulated. Any of the above methods may be automated, or may utilize tooling or jigs to provide efficient and precise processing.

In any of the methods chosen for the second annealing step, the second annealing step is, in one embodiment, performed at a temperature of about 485° C. to about 600° C. for about zero to about 120 minutes. In an exemplary embodiment, the second annealing step is performed at a temperature of about 550° C. to about 600° C. for about 5 to about 20 minutes. At such temperatures, the stiffness of the material is reduced. As with the first annealing step, the exact time and temperature of the second step depends on the material chosen. In some cases, it may be desirable for the second step to be carried out over shorter or longer periods of time and at lower or higher temperatures than those described above. In an alternative embodiment, local heat treatment the tube is performed during the second annealing step to destroy the shape memory feature of the metal in the treated region by treating the desired portions of the tube at temperatures of about 650° C. to about 700° C. and above. At temperatures of about 600° C. to about 650° C., whether the heat treatment destroys the shape memory feature depends on the duration of the treatment and the composition of the material. In a further alternative embodiment, the second annealing step is performed at a temperature of about 485° C. for about 120 minutes.

FIG. 3 illustrates a stent architecture after the stent has been cut from the tube by any method known in the art. In one embodiment, a laser cutting method is used and the cutting step is performed simultaneously with or immediately following the second annealing step.

In one embodiment, nitinol has a composition of about 55.75 weight percent nickel and 44.25 weight percent titanium. In alternative embodiments, other grades of nitinol are utilized that have different percentages of nickel and titanium, and other types of materials may also be suitable, without limitation. In addition to binary shape-memory metals such as nitinol or other alloys of nickel and titanium, the described endoluminal devices and methods are applicable for use with doped binary metals. Suitable dopants include chromium, niobium, and vanadium. Additionally, it is contemplated that other known suitable shape-memory alloys will be utilized in accordance with the teachings provided herein.

As shown in FIG. 3, a resulting device, stent 100, formed from tube 10 (shown in FIG. 2) has a plurality of sections 112 (shown highlighted in dark) that remain superelastic and allow the device to self-expand and a plurality of sections 114 that are plastically deformable and malleable to allow stent 100 to conform to a shape of the hollow organ being treated. The deformable sections may be expanded by balloon inflation from inside the device. External compression forces are typically applied to compress the stem and reduce its diameter so that it can be mounted inside a delivery system for introduction into a body lumen. When stent 100 is crimped, plastically deformable sections 114 remain crimped even after external compression forces are released. Although stent 100 may have different forms, when expanded freely outside the body or inside a cylindrical tube, it typically has a cylindrical shape. When expanded inside the body, however, stent 100 may be molded using a partially elastomeric balloon so that it conforms to the shape of the tubular structure into which it is deployed.

A known application of stents is to keep blood vessels open after angioplasty and to treat aneurysms or dissections of the blood vessels in conjunction with fabric grafts including an endograft. Iliac artery stenting is widely accepted as a treatment of the iliac artery. To treat stenosis, the artery is usually approached from the ipsilateral or contralateral common femoral artery. An 18-gauge needle is typically inserted into the artery in a retrograde fashion and a “soft tip” guidewire is advanced into the artery under fluoroscopic guidance. The needle is then removed leaving the wire in position and an introducer is placed inside the artery following the guidewire, in what is known as an “over the wire” system.

Once the wire is properly positioned, the dilator is removed and appropriate doses of heparin are injected in the vessel. Based on a pre-procedural angiogram, the most convenient x-ray incidence is chosen to obtain a new arteriogram, which is used as a road-map for the procedure. The stenosis is individualized and crossed by the guidewire, taking precaution to prevent damage to the artery. At this time, a decision is typically made regarding whether to pre-dilate the artery before placing the stent, or to proceed with “primary stenting” of the artery (stenting without pre-dilation). The lumen into which the stent is deployed should be of sufficient diameter to allow passage of a balloon guided by the guidewire after deployment of the stent. If the constriction is predicted to be hard to be dilated, for example, due to extensive calcification, balloon pre-dilation is typically performed prior to deployment of the stent. In cases of aneurysms, the initial diameter of the stent once deployed should be larger than the diameter of the neck of the aneurysm to prevent migration of the device before the final balloon dilation is applied.

The stent is inserted by advancing the stent within a delivery system into the lumen of the artery, guided by the road-map previously obtained. Once properly positioned, the stent is typically delivered by distally retracting an external sheath that radially constricts the stent. In one embodiment, the stent is prevented from retracting with the sheath by using a pusher placed distally of the stent. Alternatively, the stent is used without a pusher or with any known delivery system. The stent self-expands to a self-expanded diameter. Once the stent has self-expanded, a partially elastomeric balloon is expanded inside the stent, to further expand the stent and give the stent the appropriate shape to conform to the treated artery without leaving gaps between the stent and the wall of the vessel.

An alternative application of stent 100 is to treat, in combination with a fabric graft, aortic aneurysms and dissections. A potential problem that occurs when repairing aneurysms and dissections is the difficulty of adapting the shape of the ends of the endograft to the shape of the vessels to prevent leakage of blood inside the aneurysm. Watertight sealing of the ends of the endografts is preferred to obtain complete exclusion of aneurysms and dissections. However, a substantial percentage of aneurysmal necks and common iliac arteries in which an endograft is mounted are not cylindrical. Typically, self-expandable stents do not adapt appropriately in all irregular necks or iliac arteries, and this is a common cause of endoleaks.

In an alternative embodiment, a further method includes attaching to stent 100, by any means known in the art, a graft 116 as an inner lining (as shown in FIG. 3) or an outer covering (not shown) for the stent. Using the stent of this invention as part of an endograft 120, the self-expandable regions 114 of stent 100 anchor it in position until stent 100 is expanded by plastically deforming regions 112 of the endograft to the shape of the lumen, such as, in particular, the angle(s) of the neck of the aneurysm or iliac artery. For dissections located at the level of the aortic arch, the curve of the aorta may be duplicated by the endograft using the deformable component of the endograft as molded by a balloon.

In a further alternative embodiment, a hybrid elastic and plastically deformable stent comprises a nitinol composition that provides the stent with a low percentage recoil after elastic expansion to a first outer diameter and plastic deformation to a second outer diameter. In one embodiment, the percentage recoil from the second outer diameter to a third outer diameter is about 16% to about 3% of the plastically deformed outer diameter after the deforming device has been removed from the stent. In other words, the third outer diameter is about 86% to about 97% of the second outer diameter. In an alternative embodiment, the percentage recoil from the second outer diameter to the third outer diameter is less than about 10% of the plastically deformed outer diameter. In other words, the third outer diameter is greater than about 90% of the second outer diameter. In a further alternative embodiment, the percentage recoil is less than about 5% of the plastically deformed outer diameter. In other words, the third outer diameter is greater than about 95% of the second outer diameter. In a still further alternative embodiment, the percentage recoil is about 3.73% of the plastically deformed outer diameter. In other words, the third outer diameter is about 96.27% of the second outer diameter.

In an exemplary embodiment, the stent is fabricated by shaping the type BB alloy into a known eight apex stent configuration. Alternatively, any stent configuration may be fabricated using alloy BB. Exemplary stent configurations are described and illustrated in U.S. Pat. Nos. 5,911,733, 5,360,443, 5,578,072, and 4,733,665, which are hereby incorporated by reference in their entirety. In addition, although it is contemplated that any known stent may be fabricated as indicated herein.

In one embodiment, the stent is fabricated by pretreating the stent at about 600° C. for a period of time from about zero minutes to about 60 minutes. The stent is then aged at about 485° C. from about zero minutes to about 120 minutes. In an alternative embodiment, the stent is fabricated by pretreating the stent at about 575° C. for a period of time from about zero minutes to about 60 minutes. The stent is then aged at about 485° C. from about zero minutes to about 120 minutes. In one embodiment, the stent is plastically expanded about 3 mm to about 17 mm. In an alternative embodiment, the stent is expanded about 4 mm to about 6 mm. In a further alternative embodiment, the stent is expanded about 4.5 mm to about 5.0 mm.

Example 1

A stent fabricated from a nitinol allow type BB (currently available from Memry Corporation, 4065 Campbell Avenue, Menlo Park, Calif.) was formed into an eight apex configuration. The formed stent was not pretreated with heat. Instead, the stent was subjected to a single heating step in which the stent was aged at approximately 485° C. in a salt pot for approximately 120 minutes. The aged stent was water quenched to stop the aging process.

The stent was formed to expand elastically and then be plastically deformable. The stent had a first outer diameter (after elastic expansion at 37° C.) of about 26.492 mm and a second outer diameter (after plastic deformation utilizing a cone mandrill beginning at austenite finish dimension) of about 31.00 mm. The stent had a final outer diameter (after recoil) of about 29.845 mm which is approximately a 3.73% recoil after plastic deformation enlargement to 31 mm from an A_{f} starting diameter of 26.492 mm. The stent had a radial force (resistive) of about 1.09 pounds per inch of stent length measured against flat plates at 37° C.

As described above, a stent that is not pre treated with heat, is aged about 120 minutes and is plastically deformed about 4.5 mm to 5.0 mm, has a percentage recoil of about 3.73% and a resistive force of about 1.09 pounds per inch of stent length. Although an eight apex stent was utilized in the example, it should be understood that the above teachings are applicable to other stent configurations as well.

A method of deploying the hybrid stent fabricated as described above includes positioning the hybrid stent on an introducer, introducing the endovascular device to the proper position within the proper vessel, allowing the endovascular device to elastically expand to a first outer diameter, and plastically deforming the endovascular device to a second outer diameter.

The above described endoluminal devices can be tailored to conform to the anatomy of the lumen in which they are deployed by plastically deforming without adversely affecting the characteristics of the device. Thus, a self-expanding device can be “fine tuned” by plastic deformation to achieve optimum sizing. This application of the above described device may be particularly useful for adapting a device to a lumen having a non-round, more oval cross-section, an application for which self-expanding devices generally are considered deficient because of their tendency to deploy with a round cross-section. Additionally, the above described endoluminal devices may also have increased x-ray visibility without adding special radiopaque markers.

While the invention has been described in terms of various specific embodiments, those skilled in the art will recognize that the invention can be practiced with modification within the scope and range of equivalents of the claims and without departing from the spirit of the invention.

What is claimed is:

1. A method for making an endoluminal device comprising at least one superelastic section and at least one plastically deformable section, the superelastic section and plastically deformable section comprising a continuous metallic structure in which the superelastic section has been thermally treated differently than the plastically deformable section, the method comprising:
selecting a metallic member to be used for constructing the endoluminal device;
heat treating at least a first portion of the metallic member in a first annealing step under a first set of conditions to set a shape memory for at least the first portion;
heat treating one or more second portions of the metallic member in a second annealing step under a second set of conditions to make the second portions plastically deformable; and
forming the metallic member into the endoluminal device such that the first portion comprises the superelastic section and the second portion comprises the plastically deformable section.

2. A method in accordance with claim 1 wherein the metallic member is a hollow rube, said heat treating one or more second portions comprises cutting the tube in a pattern to form the endoluminal device.

3. A method in accordance with claim 1 wherein the endoluminal device comprises a stent.

4. A method in accordance with claim 1 wherein the metallic member comprises a shape-memory material.

5. A method in accordance with claim 1 wherein the metallic member comprises a binary metallic material.

6. A method in accordance with claim 1 wherein the metallic material comprises nickel and titanium.

7. A method in accordance with claim 1 wherein the metallic material is doped with at least one of chromium, niobium, and vanadium.

8. A method in accordance with claim 1 wherein the first portion comprises the entire metallic member.

9. A method in accordance with claim 1 wherein the first set of conditions comprises an annealing temperature ranging from about 400° C. to about 600° C. and an annealing time of about zero to about 60 minutes.

10. A method in accordance with claim 9 wherein the annealing temperature ranges from about 450° C. to about 550° C.

11. A method in accordance with claim 9 wherein the annealing temperature is about 575° C. to about 600° C.

12. A method in accordance with claim 9 wherein the annealing time ranges from about 10 minutes to about 15 minutes.

13. A method in accordance with claim 1 wherein the one or more second portions comprises one or more vertical stripes, one or more horizontal stripes, one or more isolated areas, or a combination thereof.

14. A method in accordance with claim 1 wherein the second annealing step comprises a localized heat treatment step performed by at least one of electrical resistance heating, inert gas jet heating, induction coil beating, laser healing, brazing, and fluidized bath heating with the second portion insulated.

15. A method in accordance with claim 1 wherein the second set of conditions comprises an annealing temperature ranging from about 450° C. to about 500° C. for a time period of about zero minutes to about 120 minutes.

16. A method in accordance with claim 1 wherein the second set of conditions comprises an annealing temperature of about 485° C. for about 120 minutes.

17. A method in accordance with claim 1 wherein the second set of conditions comprises an annealing temperature greater than about 650° C.

18. A method in accordance with claim 1 wherein the second set of conditions comprises an annealing temperature of about 550° C. to about 600° C. and an annealing time of about 5 minutes to about 20 minutes.

19. A method in accordance with claim 1 wherein forming the metallic member into the endoluminal device comprises a laser cutting technique or chemical etching.

20. A method in accordance with claim 1 further comprising attaching a graft as an inner liner or outer covering of the device.

21. An endovascular device configured to:
elastically expand to a first outer diameter;
plastically deform to a second outer diameter; and
retain a third outer diameter that is greater than about 90% of the second outer diameter after a device that has been utilized to deform the endovascular device to the second outer diameter has been removed from the endovascular device.

22. A device in accordance with claim 21 wherein said endovascular device retains a third outer diameter that is greater than about 95% of the second outer diameter after the deforming device has been removed from said endovascular device.

23. A device in accordance with claim 21 wherein said endovascular device retains a third outer diameter that is about 96.27% of the second outer diameter after the deforming device has been removed from said endovascular device.

24. A device in accordance with claim 21 wherein said endovascular device comprises a stent.

25. A device in accordance with claim 24 wherein said stent has a resistive force of about 1.09 pounds per inch of stent length.

26. A method of heat treating an endovascular device to form a hybrid device that is both elastic and plastically deformable, said method comprising aging the endovascular device at about 485° C. for about 120 minutes.

27. A method in accordance with claim 26 wherein the endovascular device is a formed device prior to aging, said step of aging the device is the only heating step during the heat treatment.

28. A method in accordance with claim 26 wherein the device is heat treated to recoil less than about 5% after plastic deformation.

29. A method in accordance with claim 26 wherein the device is heat treated in a salt pot.

30. A method of deploying a hybrid endovascular device fabricated from a single composition and that has undergone a single heat treatment, said method comprising:
positioning a hybrid endovascular device on an introducer;
introducing the endovascular device to the proper position;
allowing the endovascular device to elastically expand to a first outer diameter; and
plastically deforming the endovascular device to a second outer diameter.

31. A method in accordance with claim 30 wherein after plastic deformation of the endovascular device and removal of the deforming device, the device has a third outer diameter that is greater than about 95% of the second outer diameter.

32. A method in accordance with claim 30 wherein the endovascular device is a stent.

33. A method in accordance with claim 30 wherein the endovascular device comprises nitinol.
